SEQUENCE LISTING (7 sequences)

SEQ ID NO: 1 - DNA, 28 bp, Artificial Sequence
  DNA, single-stranded, artificial
tgcccattgcggaggaggaggacattgc

SEQ ID NO: 2 - DNA, 27 bp, Artificial Sequence
  DNA, single-stranded, artificial
aatgcaatgtgtggtagcaatggatcc

SEQ ID NO: 3 - DNA, 28 bp, Artificial Sequence
  DNA, single-stranded, artificial
tgcccattgcggaggaggaggacattgc

SEQ ID NO: 4 - DNA, 27 bp, Artificial Sequence
  DNA, single-stranded, artificial
aatgcaatgtgtggtagcaatggatcc

SEQ ID NO: 5 - DNA, 18 bp, Artificial Sequence
  DNA, single-stranded, artificial
tcctcctcctccgcaatg

SEQ ID NO: 6 - DNA, 13 bp, Artificial Sequence
  DNA, single-stranded, artificial
ggcaaatgcaatg

SEQ ID NO: 7 - DNA, 13 bp, Artificial Sequence
  DNA, single-stranded, artificial
tgtggtagcaatg

(19) United States
(12) Patent Application Publication
(10) Pub. No.: US 2024/0209414 A1
(43) Pub. Date: Jun. 27, 2024

(54) NOVEL NUCLEIC ACID TEMPLATE STRUCTURE FOR SEQUENCING

(71) Applicant: Roche Sequencing Solutions, Inc., Pleasanton, CA (US)

(72) Inventors: Aruna Ayer, Santa Clara, CA (US); Ni-Ting Chiou, Tanzih Township (TW)

(21) Appl. No.: 17/905,784

(22) PCT Filed: Mar. 10, 2021
(86) PCT No.: PCT/EP2021/056056

§ 371 Date: Sep. 7, 2022

Related U.S. Application Data
(60) Provisional application No. 62/988,331, filed on Mar. 11, 2020.

(51) Int. Cl. C12Q 1/6806; C12Q 1/6869

(52) U.S. Cl. CPC C12Q 1/6806; C12Q 1/6869

(57) ABSTRACT

Disclosed is a novel structure of a nucleic acid template and the method of making and using the structure. The structure consists of a double-stranded circle with a single-stranded gap. The circular gapped structure includes an extendable end from which copying or sequencing can be initiated.

### FIELD OF THE INVENTION

The invention relates to the field of nucleic acid sequencing. More specifically, the invention relates to the field of forming templates of nucleic acid targets for sequencing.

### BACKGROUND OF THE INVENTION

The wide-spread use of nucleic acid sequencing is increasing due to the development of new technologies and decreasing cost. Currently, the popular methods of high-throughput single molecule sequencing include the Illumina platforms (Illumina, San Diego, Cal.), Ion Torrent platform (Life Technologies, Grand Island, NY), Pacific BioSciences platform utilizing the SMRT (Pacific Biosciences, Menlo Park, Cal.) or a platform utilizing nanopore technology such as those manufactured by Oxford Nanopore Technologies (Oxford, UK) or Roche Sequencing Solutions (Santa Clara, Cal.).

A key to accurate long-range sequencing is the design of the nucleic acid template. Circular templates are especially advantageous for methods that do not involve cluster or polony formation but rely instead on forming a temple-polymerase complex in which the same template molecule is sequenced through a substantial length and multiple times. A circular template offers an advantage of generating a consensus from several continuous reads of the same molecule. For example, nucleic acid sequencing using biological and solid-state nanopores is a rapidly growing field, see Ameur, et al. (2019) Single molecule sequencing: towards clinical applications, Trends Biotech., 37:72, involving a biological nanopore U.S. Pat. No. 8,461,854, U.S. Ser. No. 10/337,060 or a solid-state nanopore U.S. Ser. No. 10/288,599, US20180038001, U.S. Ser. No. 10/364,507, or a tunneling junction between two electrodes PCT/EP2019/066199 and US20180217083. There is a need for innovative and economic means of forming a circular nucleic acids template for single molecule sequencing.

### SUMMARY OF THE INVENTION

The invention comprises a novel structure of a nucleic acid template for sequencing. The structure is a double-stranded circle with a short single stranded gap (“gapped circle”). The structure comprises an extendable 3′-end from which sequencing or replication can be initiated. The invention further comprises a method of using the novel template structure in sequencing as well as a method of making the novel template. The novel template is made by introducing nicks into only one strand of a double-stranded circle. The nicks are created by a nicking enzyme recognizing its specific binding sequence or by a glycosylase recognizing uracil bases in combination with a second enzyme forming a single-stranded break (nick).

In some embodiments, the invention is a method of forming a gapped circle nucleic acid template, the method comprising attaching an adaptor to at least one end of a double stranded nucleic acid in a sample forming an adapted nucleic acid, wherein only one strand of the adaptor comprises a cleavage site; joining the ends of the adapted nucleic acid to form a circular adapted nucleic acid; and contacting the circular adapted nucleic acid with a cleaving agent recognizing the cleavage site to remove a portion of only one strand in the circular adapted nucleic acid thereby forming a gapped circle nucleic acid template having a circular strand and a gapped strand. The adaptor can be attached by extending a primer comprising a target specific sequence and the adaptor sequence or by ligation. The adaptor may comprise a nucleic acid barcode.

In some embodiments, the cleaving agent is a nicking endonuclease and the cleavage site is the nicking endonuclease recognition site. In other embodiments, the cleaving agent is uracil-N-DNA glycosylase and the cleavage site is a uridine-containing nucleotide.

In some embodiments, the method further comprises a step of amplifying the adapted nucleic acid prior to forming the circular adapted nucleic acid.

In some embodiments, the method further comprises a step of contacting the sample with an exonuclease after the step of forming the circular adapted nucleic acid.

In some embodiments, the ends of the adapted nucleic acid are linked by ligation.

In some embodiments, the step of removing the portion of only one strand in the circular adapted nucleic acid is by heat denaturation after cleavage with the cleaving agent.

In some embodiments, the circular strand comprises a primer-binding site in the gap portion of the gapped circle and the method further comprises a step of annealing a primer to the primer-binding site in the circular strand and attaching the primer to the gapped strand of the gapped circle. The primer may comprises a blocking group in the 5′-portion. The blocking group may be a capture moiety and further comprising a step of capturing the gapped circle nucleic acid template by capturing the capture moiety with a capture molecule. The blocking group may be a chemical group preventing threading of the template into a nanopore, such as a hairpin structure, or a bulky group selected from a poly-cationic group, a bulky group or a base-modified nucleoside, where a poly-cationic group or a bulky group is attached to the nucleobase of the nucleoside.

In some embodiments, the gapped strand of the gapped circle comprises an extendable 3′-end and the method further comprises a step of sequencing the target nucleic acid by extending the extendable 3′-end to copy at least a portion of the circular strand.

In some embodiments, the invention is a method of sequencing nucleic acids in a sample, the method comprising, forming a library of gapped circle nucleic acid templates, the method comprising attaching an adaptor to at least one end of double stranded nucleic acids in a sample forming adapted nucleic acids, wherein only one strand of the adaptor comprises a cleavage site and the adaptor comprises a primer binding site; joining the ends of each of the adapted nucleic acids to form circular adapted nucleic acids; contacting the circular adapted nucleic acids with a cleaving agent recognizing the cleavage site to remove a portion of only one strand in each of the circular adapted nucleic acids thereby forming a library of gapped circle nucleic acid templates having a gapped strand with an extendable 3′-end and a circular strand; extending the extendable 3′-end to copy at least a portion of the circular strand thereby sequencing the library of gapped circle nucleic acid templates by a sequencing-by-synthesis method. The method may further comprise a step of enriching the nucleic acid templates prior to sequencing. During sequencing, the 3′-end is extended to copy the circular strand multiple times and the sequencing comprises a step of determining a consensus sequence by comparing multiple reads derived from extending the 3′-end to copy the circular strand multiple times and optionally, also by comparing consensus sequences of complementary strands sequenced by a method described herein.

In some embodiments, the invention is a method of forming a library of gapped circle nucleic acid templates, the method comprising: attaching an adaptor to at least one end of double stranded nucleic acids in a sample forming adapted nucleic acids, wherein one strand of the adaptor comprises a cleavage site; joining the ends of each of the adapted nucleic acids to form circular adapted nucleic acids; contacting the circular adapted nucleic acids a cleaving agent recognizing the cleavage site to remove a portion of only one strand in each of the circular adapted nucleic acids thus forming a library of gapped circle nucleic acid templates.

In some embodiments, the invention is a method of forming an enriched library of gapped circle nucleic acid templates, the method comprising: attaching an adaptor to at least one end of double stranded nucleic acids in a sample forming adapted nucleic acids, hybridizing to adapted nucleic acids a first target-specific primer having a capture moiety; capturing the adapted nucleic acid hybridized to the first primer via the capture moiety thereby enriching the target nucleic acids; hybridizing to the enriched adapted target nucleic acids a second primer comprising a sequence of one or more cleavage sites; extending the second primer to form a double-stranded adapted nucleic acid with one or more cleavage sites on only one strand; joining the ends of each of the double-stranded adapted nucleic acid to form circular adapted nucleic acids; contacting the circular adapted nucleic acids from with a cleaving agent recognizing the cleavage site to remove a portion of only one strand in each of the circular adapted nucleic acids thereby forming a library of enriched gapped circle nucleic acid templates. The method may further comprise a step of extending the first primer prior to capturing the capture moiety.

In some embodiments, the invention is a method of forming an enriched library of gapped circle nucleic acid templates, the method comprising: attaching an adaptor to at least one end of double stranded nucleic acids in a sample forming adapted nucleic acids, hybridizing to adapted nucleic acids a first target-specific primer having a capture moiety; capturing the adapted nucleic acid hybridized to the first primer via the capture moiety; hybridizing to the captured adapted nucleic acid a second primer, wherein second primer hybridizes to the same strand as the first primer; extending the hybridized second primer, thereby producing a double-stranded adapted nucleic acid and displacing the first primer comprising the capture moiety; hybridizing to the adapter within the adapted nucleic acids hybridized to the second primer a third primer comprising a sequence of one or more cleavage sites; extending the third primer forming a double-stranded adapted nucleic acid with one or more cleavage sites; joining the ends of each of the double-stranded adapted nucleic acid with one or more cleavage sites to form circular adapted nucleic acids; contacting the circular adapted nucleic acids from with a cleaving agent recognizing the cleavage site to remove a portion of one strand in each of the circular adapted nucleic acids thereby forming a library of enriched gapped circle nucleic acid templates. The first primer may be extended prior to capturing the capture moiety.

### BRIEF DESCRIPTION OF THE DRAWINGS

FIG. 1 illustrates a general scheme of forming a double-stranded gapped circle.

FIG. 2 illustrates a method of forming a double-stranded gapped circle where the nicking sites are enzyme recognition sequences introduced via tailed PCR primers.

FIG. 3 illustrates a method of forming a double-stranded gapped circle where the nicking sites are uracils introduced via tailed PCR primers.

FIG. 4 shows the products of circle formation analyzed by gel electrophoresis.

FIG. 5 shows the products of gapped circle formation analyzed by restriction enzyme digestion and gel electrophoresis.

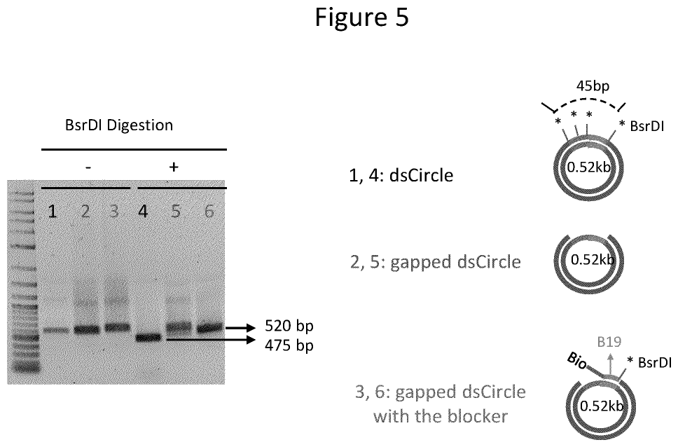

FIG. 6 illustrates a workflow including an adaptor ligation and a primer extension.

FIG. 7 illustrates a method of forming a double-stranded gapped circle with an additional step of target enrichment.

### DETAILED DESCRIPTION OF THE INVENTION

### Definitions

Unless defined otherwise, technical and scientific terms used herein have the same meaning as commonly understood by a person of ordinary skill in the art. See, Sambrook et al., Molecular Cloning, A Laboratory Manual, 4th Ed. Cold Spring Harbor Lab Press (2012).

The following definitions are provided to facilitate understanding of the present disclosure.

The term “adaptor” refers to a nucleotide sequence that may be added to another sequence in order to import additional elements and properties to that sequence. The additional elements include without limitation: barcodes, primer binding sites, capture moieties, labels, secondary structures.

The term “barcode” refers to a nucleic acid sequence that can be detected and identified. Barcodes can generally be 2 or more and up to about 50 nucleotides long. Barcodes are designed to have at least a minimum number of differences from other barcodes in a population. Barcodes can be unique to each molecule in a sample or unique to the sample and be shared by multiple molecules in the sample. The term “multiplex identifier,” “MID” or “sample barcode” refer to a barcode that identifies a sample or a source of the sample. As such, all or substantially all, MID barcoded polynucleotides from a single source or sample will share an MID of the same sequence; while all, or substantially all (e.g., at least 90% or 99%), MID barcoded polynucleotides from different sources or samples will have a different MID barcode sequence. Polynucleotides from different sources having different MIDs can be mixed and sequenced in parallel while maintaining the sample information encoded in the MID barcode. The term “unique molecular identifier” or “UID,” refer to a barcode that identifies a polynucleotide to which it is attached. Typically, all, or substantially all (e.g., at least 90% or 99%), UID barcodes in a mixture of UID barcoded polynucleotides are unique.

The term “DNA polymerase” refers to an enzyme that performs template-directed synthesis of polynucleotides from deoxyribonucleotides. DNA polymerases include prokaryotic Pol I, Pol II, Pol III, Pol IV and Pol V, eukaryotic DNA polymerase, archaeal DNA polymerase, telomerase and reverse transcriptase. The term “thermostable polymerase,” refers to an enzyme that is stable to heat, is heat resistant, and retains sufficient activity to effect subsequent polynucleotide extension reactions and does not become irreversibly denatured (inactivated) when subjected to the elevated temperatures for the time necessary to effect denaturation of double-stranded nucleic acids. A thermostable polymerase is used for amplification of nucleic acids requiring thermocycling, e.g., PCR.

In some embodiments, the polymerase has properties suitable for sequencing by synthesis and in particular, properties suitable for chip-based polynucleotide sequencing utilizing a nanopore as described in WO2013/188841. A non-limiting example of such a polymerase is described in U.S. patent Ser. No. 10/308,918. The desired characteristics of a polymerase that finds use in sequencing DNA include without limitation, slow k_{off} (for modified nucleotide), fast kₒₙ (for modified nucleotide), high fidelity, low or absent exonuclease activity, strand displacement activity, faster K_{chem} (for modified nucleotide substrates), increased stability, processivity, sequencing accuracy and long read lengths, i.e., long continuous reads. In the context of the instant invention, the strand displacement activity is required. The strand displacement activity can be experimentally determined by a displacement assay described in U.S. Ser. No. 10/308,918. The assay characterizes the ability of a polymerase unwind and displace double-stranded DNA.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologues, SNPs, and complementary sequences as well as the sequence explicitly indicated.

The term “primer” refers to an oligonucleotide, which binds to a specific region of a single-stranded template nucleic acid molecule. The oligonucleotide may be used to initiate nucleic acid synthesis via a polymerase-mediated enzymatic reaction. Typically, a primer comprises fewer than about 100 nucleotides and preferably comprises fewer than about 30 nucleotides. A target-specific primer specifically hybridizes to a target polynucleotide under hybridization conditions. Such hybridization conditions can include, but are not limited to, hybridization in isothermal amplification buffer (20 mM Tris-HCl, 10 mM (NH₄)₂SO₄), 50 mM KCl, 2 mM MgSO₄, 0.1% TWEEN® 20, pH 8.8 at 25° C.)at a temperature of about 40° C. to about 70° C. In addition to the target-binding region, a primer may have additional regions, typically at the 5′-portion. The additional region may include universal primer binding site or a barcode. Any other sequence or sequence element can be introduce via the 5′-tail sometimes referred to as the 5′-handle. The primer may also be used for purposes other than strand synthesis, e.g., to introduce an element into a nucleic acid molecule by virtue of hybridizing to a specific site in the nucleic acid molecule.

The term “sample” refers to any biological sample that comprises nucleic acid molecules, typically comprising DNA or RNA. Samples may be tissues, cells or extracts thereof, or may be purified samples of nucleic acid molecules. The term “sample” refers to any composition containing or presumed to contain target nucleic acid. Use of the term “sample” does not necessarily imply the presence of target sequence among nucleic acid molecules present in the sample. The sample can be a specimen of tissue or fluid isolated from an individual for example, skin, plasma, serum, spinal fluid, lymph fluid, synovial fluid, urine, tears, blood cells, organs and tumors, and also to samples of in vitro cultures established from cells taken from an individual, including the formalin-fixed paraffin embedded tissues (FFPET) and nucleic acids isolated therefrom. A sample may also include cell-free material, such as cell-free blood fraction that contains cell-free DNA (cfDNA) or circulating tumor DNA (ctDNA). The sample can be collected from a non-human subject or from the environment.

The term “target” or “target nucleic acid” refer to the nucleic acid of interest in the sample. The sample may contain multiple targets as well as multiple copies of each target.

The term “universal primer” refers to a primer that can hybridize to a universal primer binding site. Universal primer binding sites can be natural or artificial sequences typically added to a target sequence in a non-target-specific manner.

A key aspect of a sequencing workflow is the nucleic acid template structure and configuration. Among the sequencing methods and instruments available today, several depend or are most suitable for a circular nucleic acid template. One popular method of creating a topologically circular nucleic acid structure involves attaching stem-loop (“dumbbell”) adaptors to the ends of a linear nucleic acid fragment (see U.S. Pat. No. 8,153,375). Disclosed herein is a novel structure comprised of a double-stranded circle with a single-stranded region (gap) referred to herein interchangeably as a gapped circle or double-stranded gapped circle.

The present invention comprises sequencing target nucleic acids from a sample. In some embodiments, the sample is derived from a subject or a patient. In some embodiments the sample may comprise a fragment of a solid tissue or a solid tumor derived from the subject or the patient, e.g., by biopsy. The sample may also comprise body fluids (e.g., urine, sputum, serum, plasma or lymph, saliva, sputum, sweat, tear, cerebrospinal fluid, amniotic fluid, synovial fluid, pericardial fluid, peritoneal fluid, pleural fluid, cystic fluid, bile, gastric fluid, intestinal fluid, or fecal samples). The sample may comprise whole blood or blood fractions where normal or tumor cells may be present. In some embodiments, the sample, especially a liquid sample may comprise cell-free material such as cell-free DNA or RNA including cell-free tumor DNA or tumor RNA. In some embodiments, the sample is a cell-free sample, e.g., cell-free blood-derived sample where cell-free tumor DNA or tumor RNA are present. In other embodiments, the sample is a cultured sample, e.g., a culture or culture supernatant containing or suspected to contain nucleic acids derived from the cells in the culture or from an infectious agent present in the culture. In some embodiments, the infectious agent is a bacterium, a protozoan, a virus or a mycoplasma.

Target nucleic acids are the nucleic acid of interest that may be present in the sample. Each target is characterized by its nucleic acid sequence. The present invention enables detection of one or more RNA or DNA targets. In some embodiments, the DNA target nucleic acid is a gene or a gene fragment (including exons and introns) or an intergenic region, and the RNA target nucleic acid is a transcript or a portion of the transcript to which target-specific primers hybridize. In some embodiments, the target nucleic acid contains a locus of a genetic variant, e.g., a polymorphism, including a single nucleotide polymorphism or variant (SNP of SNV), or a genetic rearrangement resulting e.g., in a gene fusion. In some embodiments, the target nucleic acid comprises a biomarker, i.e., a gene whose variants are associated with a disease or condition. For example, the target nucleic acids can be selected from panels of disease-relevant markers described in U.S. patent application Ser. No. 14/774,518 filed on Sep. 10, 2015. Such panels are available as AVENIO ctDNA Analysis kits (Roche Sequencing Solutions, Pleasanton, Cal.) In other embodiments, the target nucleic acid is characteristic of a particular organism and aids in identification of the organism or a characteristic of the pathogenic organism such as drug sensitivity or drug resistance. In yet other embodiments, the target nucleic acid is a unique characteristic of a human subject, e.g., a combination of HLA or KIR sequences defining the subject's unique HLA or KIR genotype. In yet other embodiments, the target nucleic acid is a somatic sequence such as a rearranged immune sequence representing an immunoglobulin (including IgG, IgM and IgA immunoglobulin) or a T-cell receptor sequence (TCR). In yet another application, the target is a fetal sequence present in maternal blood, including a fetal sequence characteristic of a fetal disease or condition or a maternal condition related to pregnancy. For example, the target could be one or more of the autosomal or X-linked disorders described in Zhang et al. (2019) Non-invasive prenatal sequencing for multiple Mendelian monogenic disorders using circulating cell-free fetal DNA, Nature Med. 25(3):439.

In some embodiments, the target nucleic acid is RNA (including mRNA, microRNA, viral RNA). In other embodiments, the target nucleic acid is DNA including cellular DNA or cell-free DNA (cfDNA) including circulating tumor DNA (ctDNA). The target nucleic acid may be present in a short or long form. Longer target nucleic acids may be fragmented. In some embodiments, the target nucleic acid is naturally fragmented, e.g., includes circulating cell-free DNA (cfDNA) or chemically degraded DNA such as the one found in chemically preserved or ancient samples.

In some embodiments, the invention comprises a step of nucleic acid isolation. Generally, any method of nucleic acid extraction that yields isolated nucleic acids comprising DNA or RNA may be used. Genomic DNA or RNA may be extracted from tissues, cells, liquid biopsy samples (including blood or plasma samples) using solution-based or solid-phase based nucleic acid extraction techniques. Nucleic acid extraction can include detergent-based cell lysis, denaturation of nucleoproteins, and optionally removal of contaminants. Extraction of nucleic acids from preserved samples may further include a step of deparaffinization. Solution based nucleic acid extraction methods may comprise salting out methods or organic solvent or chaotrope methods. Solid-phase nucleic extraction methods can include but are not limited to silica resin methods, anion exchange methods or magnetic glass particles and paramagnetic beads (KAPA Pure Beads, Roche Sequencing Solutions, Pleasanton, Cal.) or AMPure beads (Beckman Coulter, Brea, Cal.)

A typical extraction method involves lysis of tissue material and cells present in the sample. Nucleic acids released from the lysed cells can be bound to a solid support (beads or particles) present in solution or in a column, or membrane where the nucleic acids may undergo one or more washing steps to remove contaminants including proteins, lipids and fragments thereof from the sample. Finally, the bound nucleic acids can be released from the solid support, column or membrane and stored in an appropriate buffer until ready for further processing. Depending on whether DNA or RNA are being isolated, an appropriate nuclease or nuclease inhibitor may be used to preferentially isolate only one type of nucleic acid. If both DNA and RNA are to be isolated, no nuclease and optionally a nuclease inhibitor may be used during the nucleic acid isolation and purification process.

In some embodiments, the input DNA or input RNA require fragmentation. In such embodiments, RNA may be fragmented by a combination of heat and metal ions, e.g., magnesium. In some embodiments, the sample is heated to 85°-94ºC for 1-6 minutes in the presence of magnesium. (KAPA RNA HyperPrep Kit, KAPA Biosystems, Wilmington, Mass). DNA can be fragmented by physical means, e.g., sonication, using available instruments (Covaris, Woburn. Mass.) or enzymatic means (KAPA Fragmentase Kit, KAPA Biosystems).

In some embodiments, the isolated nucleic acid is treated with DNA repair enzymes. In some embodiments, the DNA repair enzymes comprise a DNA polymerase which has 5′-3′ polymerase activity and 3′-5′ single stranded exonuclease activity, a polynucleotide kinase which adds a 5′ phosphate to the dsDNA molecule, and a DNA polymerase which adds a single dA base at the 3′ end of the dsDNA molecule. The end repair/A-tailing kits are available e.g., Kapa Library Preparation, kits including KAPA Hyper Prep and KAPA HyperPlus (Kapa Biosystems, Wilmington, Mass.).

In some embodiments, the DNA repair enzymes target damaged bases in the isolated nucleic acids. In some embodiments, sample nucleic acid is partially damaged DNA from preserved samples, e.g., formalin-fixed paraffin embedded (FFPET) samples. Deamination and oxidation of bases can result in an erroneous base read during the sequencing process. In some embodiments, the damaged DNA is treated with uracil N-DNA glycosylase (UNG/UDG) and/or 8-oxoguanine DNA glycosylase.

In some embodiments, the invention utilizes an adaptor nucleic acid. The adaptor may be added to the nucleic acid by a blunt-end ligation or a cohesive end ligation. In some embodiments, the adaptor may be added by single-strand ligation method. In some embodiments, the adaptor molecules are in vitro synthesized artificial sequences. In other embodiments, the adaptor molecules are in vitro synthesized naturally occurring sequences. In yet other embodiments, the adaptor molecules are isolated naturally occurring molecules or isolated non-naturally occurring molecules.

In the case of adaptor added by ligation, the adaptor oligonucleotide can have overhangs or blunt ends on the terminus to be ligated to the target nucleic acid. In some embodiments, the adaptor comprises blunt ends to which a blunt-end ligation of the target nucleic acid can be applied. The target nucleic acids may be blunt-ended or may be rendered blunt-ended by enzymatic treatment (e.g., “end repair.”). In other embodiments, the blunt-ended DNA undergoes A-tailing where a single A nucleotide is added to the 3′-end of one or both blunt ends. The adaptors described herein are made to have a single T nucleotide extending from the blunt end to facilitate ligation between the nucleic acid and the adaptor. Commercially available kits for performing adaptor ligation include AVENIO ctDNA Library Prep Kit or KAPA HyperPrep and HyperPlus kits (Roche Sequencing Solutions, Pleasanton, Cal.). In some embodiments, the adaptor ligated DNA may be separated from excess adaptors and unligated DNA.

In some embodiments, the adaptor contains one or more novel elements described herein including a nicking endonuclease recognition sequence or deoxyuracils. The adaptor may further comprise features such as universal primer binding site (including a sequencing primer binding site) a barcode sequence (including a sample barcode (SID) or a unique molecular barcode or identifier (UID or UMI). In some embodiments, the adaptors comprise all of the above features while in other embodiments, some of the features are added after adaptor ligation by extending tailed primers that contain some of the elements described above.

The adaptor may further comprise a capture moiety. The capture moiety may be any moiety capable of specifically interacting with another capture molecule. Capture moieties—capture molecule pairs include avidin (streptavidin)—biotin, antigen—antibody, magnetic (paramagnetic) particle—magnet, or oligonucleotide—complementary oligonucleotide. The capture molecule can be bound to a solid support so that any nucleic acid on which the capture moiety is present is captured on solid support and separated from the rest of the sample or reaction mixture. In some embodiments, the capture molecule comprises a capture moiety for a secondary capture molecule. For example, a capture moiety in the adaptor may be a nucleic acid sequence complementary to a capture oligonucleotide. The capture oligonucleotide may be biotinylated so that adapted nucleic acid-capture oligonucleotide hybrid can be captured on a streptavidin bead.

In some embodiments, the invention utilizes a barcode. Detecting individual molecules typically requires molecular barcodes such as described in U.S. Pat. Nos. 7,393,665, 8,168,385, 8,481,292, 8,685,678, and 8,722,368. A unique molecular barcode is a short artificial sequence added to each molecule in the patient's sample typically during the earliest steps of in vitro manipulations. The barcode marks the molecule and its progeny. The unique molecular barcode (UID) has multiple uses. Barcodes allow tracking each individual nucleic acid molecule in the sample to assess, e.g., the presence and amount of circulating tumor DNA (ctDNA) molecules in a patient's blood in order to detect and monitor cancer without a biopsy (Newman, A., et al., (2014) An ultrasensitive method for quantitating circulating tumor DNA with broad patient coverage, Nature Medicine doi:10.1038/nm.3519).

A barcode can be a multiplex sample ID (MID) used to identify the source of the sample where samples are mixed (multiplexed). The barcode may also serve as a unique molecular ID (UID) used to identify each original molecule and its progeny. The barcode may also be a combination of a UID and an MID. In some embodiments, a single barcode is used as both UID and MID. In some embodiments, each barcode comprises a predefined sequence. In other embodiments, the barcode comprises a random sequence. In some embodiments of the invention, the barcodes are between about 4-20 bases long so that between 96 and 384 different adaptors, each with a different pair of identical barcodes are added to a human genomic sample. A person of ordinary skill would recognize that the number of barcodes depends on the complexity of the sample (i.e., expected number of unique target molecules) and would be able to create a suitable number of barcodes for each experiment.

Unique molecular barcodes can also be used for molecular counting and sequencing error correction. The entire progeny of a single target molecule is marked with the same barcode and forms a barcoded family. A variation in the sequence not shared by all members of the barcoded family is discarded as an artifact and not a true mutation. Barcodes can also be used for positional deduplication and target quantification, as the entire family represents a single molecule in the original sample (Newman, A., et al., (2016) Integrated digital error suppression for improved detection of circulating tumor DNA, Nature Biotechnology 34:547).

In some embodiments, the number of UIDs in the plurality of adaptors may exceed the number of nucleic acids in the plurality of nucleic acids. In some embodiments, the number of nucleic acids in the plurality of nucleic acids exceeds the number of UIDs in the plurality of adaptors.

In some embodiments, the invention further includes a structure and method preventing threading of the template into a nanopore during sequencing. This is especially advantageous for sequencing methods that utilize a nanopore but do not involve threading of any nucleic acid into the nanopore. (see e.g. U.S. Pat. No. 8,461,854).

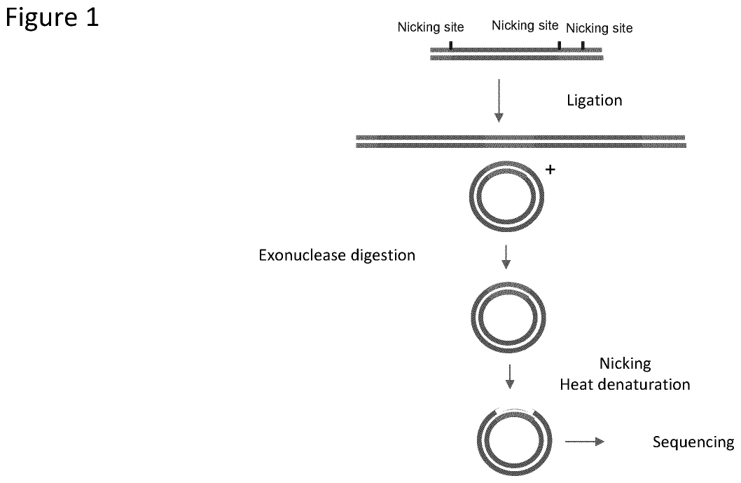

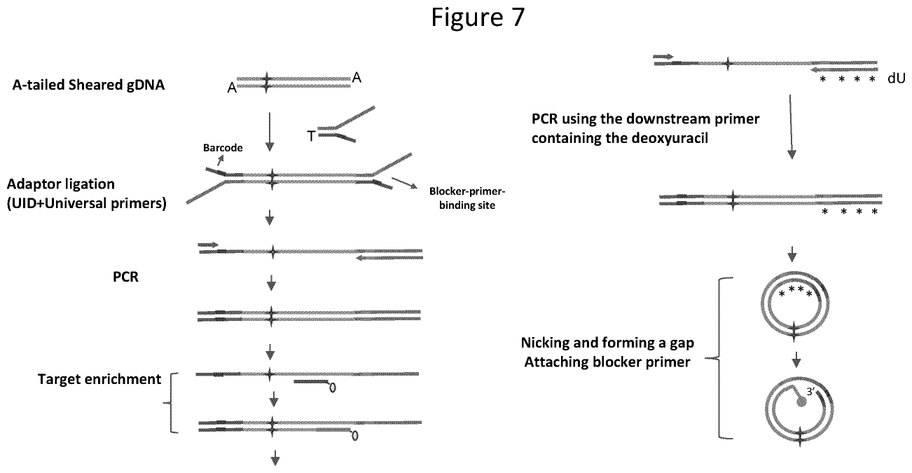

In this embodiment, the method includes a step of inserting a threading prevention structure into the gap portion of the gapped circled formed as describe herein. Specifically, an oligonucleotide primer may bind to a binding site in the gap. The binding site for the primer is incorporated into the gapped circle nucleic acid template by virtue of being present in the adaptor. (see FIGS. 1, 2 and 3 and especially FIG. 7).

In some embodiments, the adaptor added to the nucleic acid template by ligation comprises primer a binding site. In other embodiments, each of the two adaptors added to the nucleic acid template by ligation comprises a portion of the primer a binding site so that upon circularization, a complete primer binding site is formed in the circular template.

In some embodiments, the adaptor added to the nucleic acid template by primer extension comprises primer a binding site. For example, one of the primers may comprise a primer binding site. In other embodiments, each of the two primers used for primer extension comprises a portion of the primer a binding site so that upon primer extension and circularization, a complete primer binding site is formed in the circular template.

The primer annealing to the primer binding site may be attached, e.g., by ligation to the gapped strand in the gapped nucleic acid template. The primer comprises a threading blocker structure at the 5′-end. Upon annealing and ligation of the primer, the gapped strand in the gapped nucleic acid template comprises a threading blocker structure at the 5′-end.

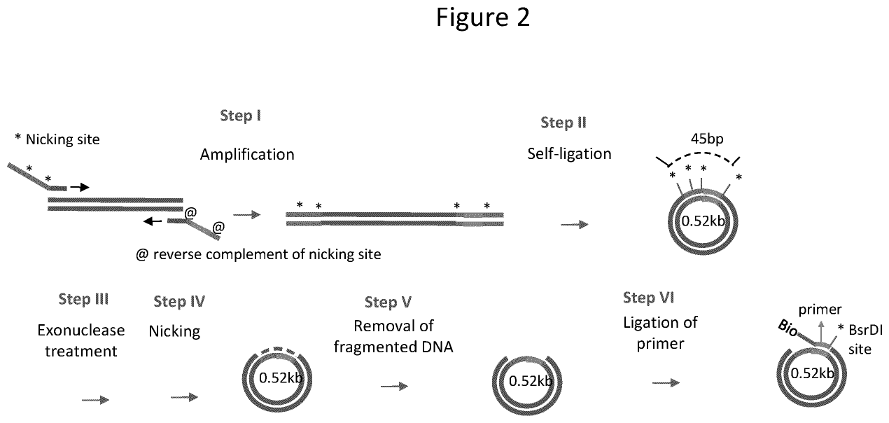

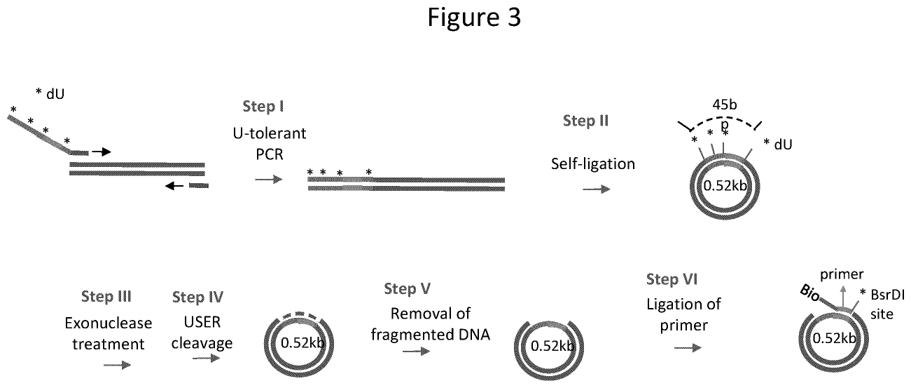

In some embodiments, the blocking structure is biotin (FIG. 2, bottom rights, FIG. 3, bottom right).

In other embodiments, the blocking structure preventing threading of the template strand into nanopore is a hairpin structure. Examples of suitable hairpin structures have been described in the U.S. provisional application Ser. No. 62/936,264 filed on Nov. 15, 2019 and titled “Structure to prevent threading of nucleic acid templates through a nanopore during sequencing.”

In other embodiments, the blocking structure preventing threading of the template strand into nanopore is a chemical moiety attached to the 5′-end of the primer and selected from a poly-cationic group, a bulky group or a base-modified nucleoside, where a poly-cationic group or a bulky group is attached to the nucleobase of the nucleoside, see e.g., the U.S. provisional application Ser. No. 62/971,078 filed on Feb. 6, 2020 and titled “Compositions that reduce template threading into a nanopore.”

In some embodiments, the invention comprises an amplification step involving linear or exponential amplification. Amplification may be isothermal or involve thermocycling. In some embodiments, the amplification is exponential and involves PCR. In some embodiments, gene-specific primers are used for amplification. In other embodiments, universal primer binding sites are added to target nucleic acid e.g., by ligating an adaptor comprising the universal primer binding sites. All adaptor-ligated nucleic acids have the same universal primer binding sites and can be amplified with the same set of primers. The number of amplification cycles where universal primers are used can be low but also can be 10, 20 or as high as about 30 or more cycles, depending on the amount of product needed for the subsequent steps. Because PCR with universal primers has reduced sequence bias, the number of amplification cycles need not be limited to avoid amplification bias.

In some embodiments, the invention involves an amplification step, e.g., prior to or after ligating adaptors or prior to or after extending 5′-tailed (“handle”) primers. The amplification primers may be target-specific. A target specific primer comprises at least a portion that is complementary to a sequence in the target. If additional sequences are present, such as a barcode, a second primer binding site or a nuclease recognition site, they are typically located in the 5′-portion of the primer. In other embodiments, the primers are universal, e.g., can amplify all nucleic acids in the sample regardless of the target sequence. Universal primers anneal to universal primer binding sites added to the nucleic acids in the sample by extending a primer having the universal primer binding site or by ligating an adaptor having a universal primer binding site.

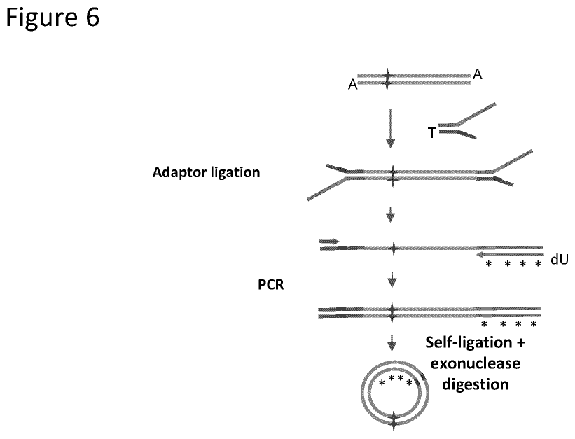

Primers may also be used as capture probes to enrich for target nucleic acids as described herein. The term primer and probe may be used interchangeably to designate a short oligonucleotide binding to its target under certain conditions. As dieclosed herein (FIG. 6) an oligonucleotide with a capture moiety can be used to enrich the target nucleic acid by retaining the captured desired nucleic acids or by depleting the captured undesired nucleic acids.

In some embodiments, the invention is a library of target nucleic acids formed as described herein. The library comprises double-stranded nucleic acid molecules comprising nucleic acid targets present in the original sample. The nucleic acid molecules of the library further comprise novel adaptors described herein at one or both ends of the target nucleic acid sequence. The library nucleic acids may comprise additional elements such as barcodes and primer binding sites. In some embodiments, the additional elements are present in adaptors and are added to the library nucleic acids via adaptor ligation. In other embodiments, some or all of the additional elements are present in amplification primers and are added to the library nucleic acids prior to adaptor ligation by extension of the primers. The amplification may be linear (including only one round of extension) or exponential, e.g., Polymerase Chain Reaction (PCR). In some embodiments, some additional elements are added by primer extension while the remaining additional elements are added by adaptor ligation.

The utility of adaptors and amplification primers for introducing additional elements in to a library of nucleic acids to be sequenced has been described e.g., in U.S. Pat. Nos. 9,476,095, 9,260,753, 8,822,150, 8,563,478, 7,741,463, 8,182,989 and 8,053,192.

In some embodiments, the invention further comprises a step of enriching for desired target nucleic acids. The desired nucleic acids can be enriched prior to forming a library according to the novel library forming method of described herein. Alternatively, the enrichment can take place after eh library is formed, i.e., on the molecules of the library.

In some embodiments, the method utilizes a pool of target-specific oligonucleotide probes (e.g., capture probes). The enrichment can be by subtraction in which case, capture probes are complementary to an abundant undesired sequences including ribosomal RNA (rRNA) or abundantly expressed genes (e.g., globin). In the case of subtraction, the undesired sequences are captured by the capture probes and removed from the mixture of target nucleic acids or the library of nucleic acids and discarded. For example, the capture probes may comprise a binding moiety that can be captured on solid support.

In other embodiments, the enrichment is capture and retention in which case, capture probes are complementary to one or more target sequences. In this case the target sequences are captured by the capture probes from the mixture of target nucleic acids or the library of nucleic acids and retained while the remainder of the solution is discarded.

For enrichment, the capture probes may be free in solution or fixed to solid support. The probes can be produced and amplified e.g., by the method described in the U.S. Pat. No. 9,790,543. The probes may also comprise a binding moiety (e.g., biotin) and be capable of being captured on solid support (e.g., avidin or streptavidin containing support material).

In some embodiments, enrichment is by Primer Extension Target Enrichment (PETE). Multiple versions of PETE are described in U.S. application Ser. Nos. 14/910,237, 15/228,806, 15/648,146 and International Application Ser. No. PCT/EP2018/085727.

Briefly, Primer Extension Target Enrichment (PETE) involves capturing nucleic acids with a first target-specific primer comprising a capture moiety and capturing the capture moiety thereby enriching the target nucleic acids. Any additional target-specific or adapter-specific primers hybridize to the enriched target nucleic acids. In other embodiments, PETE involves capturing nucleic acids by hybridizing and extending a first primer comprising a capture moiety and capturing the capture moiety thereby enriching the target nucleic acids, hybridizing to the captured nucleic acids a second target-specific primer, extending the second target-specific primer thereby displacing the extension product of the first target-specific primer and further enriching the target nucleic acid.

Enrichment may utilize a capture moiety. A capture moiety may be any moiety capable of specifically interacting with another capture molecule. Capture moieties—capture molecule pairs include avidin (streptavidin)—biotin, antigen—antibody, magnetic (paramagnetic) particle—magnet, or oligonucleotide complementary oligonucleotide. The capture molecule can be bound to a solid support so that any nucleic acid on which the capture moiety is present is captured on solid support and separated from the rest of the sample or reaction mixture. In some embodiments, the capture molecule comprises a capture moiety for a secondary capture molecule. For example, a capture moiety may be an oligonucleotide complementary to a capture oligonucleotide (capture molecule). The capture oligonucleotide may be biotinylated and captured on a streptavidin bead.

In some embodiments, the adaptor-ligated nucleic acid is enriched via capturing the capture moiety and separating the adaptor-ligated target nucleic acids from unligated nucleic acids in the sample.

In some embodiments, the third oligonucleotide hybridized to the 3′-end of the bottom adaptor strand serves as a sequencing primer or an amplification primer. In some embodiments, the extension product of the third oligonucleotide is captured via the capture moiety. Capture of the extension product separates the extension product from unligated sample nucleic acids and optionally, from the target nucleic acids strands not having the capture moiety as well.

In some embodiments, the stem portion of the adaptor includes a modified nucleotide increasing the melting temperature of the capture oligonucleotide, e.g., 5-methyl cytosine, 2,6-diaminopurine, 5-hydroxybutynl-2′-deoxyuridine, 8-aza-7-deazaguanosine, a ribonucleotide, a 2′O-methyl ribonucleotide or a locked nucleic acid. In another aspect, the capture oligonucleotide is modified to inhibit digestion by a nuclease, e.g., by a phosphorothioate nucleotide.

In some embodiments, the invention comprises intermediate purification steps. For example, any unused oligonucleotides such as excess primers and excess adaptors are removed, e.g., by a size selection method selected from gel electrophoresis, affinity chromatography and size exclusion chromatography. In some embodiments, size selection can be performed using Solid Phase Reversible Immobilization (SPRI) technology from Beckman Coulter (Brea, Cal.). In some embodiments, a capture moiety (FIG. 2) is used to capture and separate adaptor-ligated nucleic acids from unligated nucleic acids or primer extension products from the template strands.

In some embodiments, unreacted linear nucleic acids, e.g., primers, probes adaptors or unligated template nucleic acids are removed from the reaction mixture by exonuclease digestion. In some embodiments, digestion with T7 exonuclease, T5 exonuclease, Lambda exonuclease, or Exonuclease I, V or VIII is used to remove the combination of unreacted linear oligonucleotides and un-circularized (linear) double-stranded adapted nucleic acid.

The invention comprises a method of forming a template suitable for sequencing by a single-molecule sequencer such as for example, a nanopore sequencer performing a sequencing-by-synthesis method. The method comprises forming a gapped circle template having a circular strand and a gapped strand. In some embodiments, the method comprises attaching an adaptor to one or both ends of a double stranded nucleic acid so that a resulting double-stranded adapted nucleic acid has cleavage sites on only one of the strands. (FIG. 1, top). For example, the adaptor sequence may be added by extending a primer with a target-specific 3′-portion or random 3′-portion and a 5′-“handle” comprising the adaptor sequence (FIG. 2, top-left, and FIG. 3, top-left). The forward primer may comprise a nicking enzyme recognition site while the reverse primer comprises a reverse complement of the recognition site. In the embodiment where the cleavage site is a deoxyuracil, only one of the forward and reverse primers comprises one or more deoxyuracils. The use of uracil-tolerant polymerase enables the use of a dU-containing primer in each round of amplification. (FIG. 3, top middle).

In some embodiments, the adaptor with the cleavage site is added by ligation to the target nucleic acid. In some embodiments, a combination strategy is used: an adaptor containing primer-binding sites is ligated to the target nucleic acid. A primer comprising a 5′-handle with one or more nicking sites is hybridized to the adapted nucleic acid and extended to form a nucleic acid with nicking sites on only one strand. (FIG. 6)

Following the introduction of the cleavage sites, the double-stranded adapted molecule is self-circularized to form a circle where only one of the strands has one or more cleavage sites. (FIG. 1, middle, FIG. 2, top-right, and FIG. 3, top-right.) In some embodiments, the self-circularization is by ligation of the two ends of the double-stranded adapted molecule. In some embodiments, the 5′-ends of the two strands in the double-stranded adapted molecule are phosphorylated in order for ligation to take place.

In some embodiments, the double-stranded adapted molecule is amplified prior to circularization.

In some embodiments, the non-circularized double-stranded adapted molecules are removed from the reaction mixture. In some embodiments, the removal is accomplished by exonuclease treatment to which only linear (non-circular) nucleic acids are susceptible. (FIG. 1, middle, FIG. 2, bottom left, and FIG. 3, bottom left). In other embodiments, circular and linear molecules are separated based on their physical properties, e.g., speed of electrophoretic migration or speed of passage through a size separation or size exclusion chromatography column.

In some embodiments, the cleavage site is a recognition site for a nicking endonuclease. For example, small subunits of some heterodimer restriction endonucleases behave as sequence-specific DNA nicking enzymes and only cleave one strand of the recognition site. Xu, et al. (2007) Discovery of natural nicking endonucleases Nb.BsrDI and Nb.BtsI and engineering of top-strand nicking variants from BsrDI and BtsI, NAR 35:4608. Other nicking enzymes with different recognition sequences have since been discovered or engineered and are commercially available (New England BioLabs, Ipswich, Mass.). In the context of the present invention, the double stranded adapted nucleic acids having a nicking enzyme site in only one strand are incubated with the corresponding nicking enzyme in a suitable buffer under manufacturer-recommended conditions to achieve cleavage and generation of one or more nicks in only one strand of the circular double-stranded adapted molecules. (FIG. 1, bottom, FIG. 2, bottom left).

In other embodiments, the cleavage site is present in only one strand of the adaptor in the form of deoxyuridine. In some embodiments, a uracil-containing adaptor is ligated to at least one end of the target nucleic acid so that uracil is present in only one strand of the circular double-stranded adapted molecules. In other embodiments, the uracil-containing adaptor is added by extending a primer comprising uracil. In some embodiments, the uracil-containing primer sequence is copied by a uracil-tolerant polymerase, e.g., Q5U DNA polymerase (New England BioLabs, Ipswich, Mass.). (FIG. 3, top left).

Uracil base can be excised from one strand of the circular double-stranded adapted molecules with a uracil-N-DNA glycosylase enzyme (UNG or UDG). The enzyme leaves an abasic site, which can cause a break in the phosphor-diester bond resulting in a nick. Formation of the nick is favored under increased temperature and (or) in the presence of amine compounds. The nick can also be introduced by treatment with an endonuclease recognizing abasic sites, e.g., Endonuclease VIII. Enzymatic reagents combining the glycosylase and endonuclease activities in a single preparation are commercially available (e.g., USER® enzyme, New England BioLabs).

The method further comprises a step of forming a gap at the site of one or more nicks in one strand of the circular double-stranded adapted molecules. In some embodiments, the distance between the outer-most cleavage sites is about 45 bases but can also be about 10, 20, 30, 40, 50 or 60 bases in lengths or any number in between. The number of cleavage sites is about one per every 10 bases or any similar distance that accommodates the size of the cleavage enzyme recognition site. (FIG. 2, top right, FIG. 3, top right). The placement of the cleavage sites results in multiple nicks (single-strand breaks in the sugar-phosphate backbone) of one strand in the double-stranded circular nucleic acid. (FIG. 2, bottom left, FIG. 3, bottom left). The nucleic acid strand fragments between the two nicks can be dissociated from the double-stranded circular nucleic acid leaving a gap in one of the strands of the double-stranded circular nucleic acid. (FIG. 1, bottom, FIG. 2, bottom center, FIG. 3, bottom center) In some embodiments, fragments resulting from nicking are separated from the circular double-stranded adapted molecules by increased temperatures in an appropriate buffer. In some embodiments, denaturation of the fragments resulting from nicking is facilitated by competition with excess oligonucleotides capable of hybridizing to the fragments to be removed.

In some embodiments, the method further comprises inserting a threading block structure into the gap of the gapped circle nucleic acid template molecule. The portion of the circular strand facing the gap may comprise a primer binding site. The method then further comprises a step of annealing or hybridizing an oligonucleotide primer to the primer binding site in the gap of the gapped circle. The primer can be ligated to the gapped strand in the gapped circle thus attaching the primer to one strand of the gapped circle. (FIG. 2, bottom right, FIG. 3, bottom right). The primer comprises an advantageous structure or modification on the 5′-end (free end, unligated to a strand of the gapped circle). In some embodiments, the modification is a capture moiety, e.g., biotin. (FIG. 2, bottom right, FIG. 3, bottom right). In some embodiments, the method further comprises capturing the gapped circle nucleic acid template by capturing the capture moiety with a capture molecule.

In some embodiments, the 5′-end modification of the primer is a chemical group preventing threading of the template into a nanopore, such as a poly-cationic group, a bulky group or a base-modified nucleoside, where a poly-cationic group or a bulky group is attached to the nucleobase of the nucleoside. In some embodiments, group preventing threading of the template into a nanopore is a hairpin structure formed by the 5′-end of the primer.

While the 5′-end of the gapped strand in the double-stranded gapped nucleic acid template may be free or may be blocked by a capture molecule or a structure preventing threading into the nanopore, the 3′-end of the gapped strand in the double-stranded gapped nucleic acid template is extendable. In some embodiments, the method further comprises a step of extending the 3′-end of the gapped strand in the double-stranded gapped nucleic acid template thereby sequencing the nucleic acid template by a sequencing by synthesis (SBS) method.

In some embodiments, the method further comprises enriching the gapped circle nucleic acid templates prior to sequencing by concentrating the nucleic acids via sie exclusion column or an affinity column.

In some embodiments, the circular nucleic acid strand is read multiple times during the sequencing by synthesis (SBS) process. The multiple reads of the sequence of the circular strand are used to determine a consensus sequence of the circular strand that is free or substantially free of sequencing errors.

In some embodiments, the templates, or libraries of templates formed according to the present invention are enriched for one or more target nucleic acids. The enrichment can be by retention, i.e., the desired sequences are captured and retained while the non-captured sequences are not retained and are optionally discarded. In other embodiments, the enrichment is by depletion, i.e., undesired sequences are captured and removed from the sample or reaction mixture while the desired sequences remain in the sample and are retained.

As illustrated in FIG. 7, in some embodiments, the method of forming an enriched library of gapped nucleic acid templates comprises a step of attaching an adaptor to at least one end of double stranded nucleic acids in a sample forming adapted nucleic acids. Next, the adapted nucleic acid is hybridized to a first target-specific primer having a capture moiety. The adapted nucleic acid hybridized to the primer is captured via the capture moiety thereby enriching the target adapted nucleic acid. In some embodiments, the capture moiety is captured by a ligand attached to a solid support. The solid support with the captured target nucleic acid is separated from the liquid phase containing the remainder of adapted nucleic acids. Following the separation, the captured nucleic acids are introduced into another reaction mixture as enriched nucleic acids.

The enriched nucleic acids a contacted with a second primer comprising a sequence of one or more cleavage sites. In some embodiments, the 3′-portion of the second primer comprises a target-specific sequence or a sequence hybridizing to the adaptor in the adapted nucleic acids. The 5′-portion of the second primer comprises a sequence with one or more cleavage sites. In some embodiments, the 5′-portion of the second primer comprises a cleavage site in the form of a recognition sequence for a nicking enzyme. In some embodiments, the cleavage site in the primer is a uracil-containing nucleotide such as uracil or deoxyuracil. In this embodiment, the 5′-portion of the second primer is optional. Instead, the thymines in the target-specific portion of the second primer are replaced with uracils.

The second primer is extended forming a double-stranded adapted nucleic acid with one or more cleavage sites on only one strand. Next, the ends of the double-stranded adapted nucleic acid are joined to form circular adapted nucleic acids with cleavage sites in only one of the strands. Next, the circular adapted nucleic acids are cleaved with a cleaving agent recognizing the cleavage sites to remove a portion of only one strand in each of the circular adapted nucleic acids thereby forming a library of enriched gapped circle nucleic acid templates.

In some embodiments, the templates, or libraries of templates formed according to the present invention are enriched for one or more target nucleic acids by a different method. This embodiment of the method of forming an enriched library of gapped nucleic acid templates comprises a step of attaching an adaptor to at least one end of double stranded nucleic acids in a sample forming adapted nucleic acids. Next, the adapted nucleic acid is hybridized to a first target-specific primer having a capture moiety. Optionally, the hybridized primer is extended to copy a strand of the target nucleic acid. The adapted nucleic acid hybridized to the primer is captured via the capture moiety thereby enriching the target adapted nucleic acid. In some embodiments, the capture moiety is captured by a ligand attached to a solid support. The solid support with the captured target nucleic acid is separated from the liquid phase containing the remainder of adapted nucleic acids. Following the separation, the captured nucleic acids are introduced into another reaction mixture.

The reaction mixture with enriched target nucleic acids is contacted with a second target-specific primer hybridizing to the target nucleic acid internally to the first target-specific primer. The method then comprises extending the hybridized second primer, thereby producing a double-stranded adapted nucleic acid and displacing the first primer (or the first primer extension product) comprising the capture moiety and releasing the target nucleic acid and the second primer extension product into solution thereby further enriching the target nucleic acid in solution.

Next, the method comprises hybridizing to the enriched nucleic acids a third primer comprising a sequence of one or more cleavage sites. In some embodiments, the 3′-portion of the third primer comprises a target-specific or adaptor-specific sequence and the 5′-portion of the third primer comprises one or more cleavage sites. In some embodiments, the cleavage site is a recognition sequence for a nicking enzyme. In some embodiments, the cleavage site is uracil or deoxyuracil, which may be placed in the target-specific or adapter-specific portion of the primer or in the additional 5′-portion of the primer.

Next, the third primer is extended forming a double-stranded adapted nucleic acid with one or more cleavage sites; and the ends of each of the double-stranded adapted nucleic acid are self-joined to form circular adapted nucleic acids. The circular adapted nucleic acids are cleaved with a cleaving agent recognizing the cleavage site to remove a portion of one strand in each of the circular adapted nucleic acids thereby forming a library of enriched gapped circle nucleic acid templates.

The nucleic acids and libraries of nucleic acids formed as described herein or amplicons thereof can be subjected to nucleic acid sequencing. Sequencing can be performed by any method known in the art. Especially advantageous is the high-throughput single molecule sequencing method utilizing nanopores. In some embodiments, the nucleic acids and libraries of nucleic acids formed as described herein are sequenced by a method involving threading through a biological nanopore (U.S. Ser. No. 10/337,060) or a solid-state nanopore (U.S. Ser. No. 10/288,599, US20180038001, U.S. Ser. No. 10/364,507). In other embodiments, sequencing involves threading tags through a nanopore. (U.S. Pat. No. 8,461,854) or any other presently existing or future DNA sequencing technology utilizing nanopores.

Other suitable technologies of high-throughput single molecule sequencing. include the Illumina HiSeq platform (Illumina, San Diego, Cal.), Ion Torrent platform (Life Technologies, Grand Island, NY), Pacific BioSciences platform utilizing the SMRT (Pacific Biosciences, Menlo Park, Cal.) or a platform utilizing nanopore technology such as those manufactured by Oxford Nanopore Technologies (Oxford, UK) or Roche Sequencing Solutions (Santa Clara, Cal.) and any other presently existing or future DNA sequencing technology that does or does not involve sequencing by synthesis. The sequencing step may utilize platform-specific sequencing primers. Binding sites for these primers may be introduced in 5′-portions of the amplification primers used in the amplification step. If no primer sites are present in the library of barcoded molecules, an additional short amplification step introducing such binding sites may be performed. In some embodiments, the sequencing step involves sequence analysis. In some embodiments, the analysis includes a step of sequence aligning. In some embodiments, aligning is used to determine a consensus sequence from a plurality of sequences, e.g., a plurality having the same barcodes (UID). In some embodiments barcodes (UIDs) are used to determine a consensus from a plurality of sequences all having an identical barcode (UID). In other embodiments, barcodes (UIDs) are used to eliminate artifacts, i.e., variations existing in some but not all sequences having an identical barcode (UID). Such artifacts resulting from PCR errors or sequencing errors can be eliminated.

In some embodiments, the number of each sequence in the sample can be quantified by quantifying relative numbers of sequences with each barcode (UID) in the sample. Each UID represents a single molecule in the original sample and counting different UIDs associated with each sequence variant can determine the fraction of each sequence in the original sample. A person skilled in the art will be able to determine the number of sequence reads necessary to determine a consensus sequence. In some embodiments, the relevant number is reads per UID (“sequence depth”) necessary for an accurate quantitative result. In some embodiments, the desired depth is 5-50 reads per UID.

In some embodiments, the step of sequencing further includes a step of error correction by consensus determination. Sequencing by synthesis of the circular strand of the gapped circular template disclosed herein enables iterative or repeated sequencing. Multiple reads of the same nucleotide position enable sequencing error correction through establishment of a consensus call for each nucleotide or for the entire sequence or for a part of the sequence. The final sequence of a nucleic acid strand is obtained from the consensus base determinations at each position. In some embodiments, a consensus sequence of a nucleic acid is obtained from a consensus obtained by comparing the sequences of complementary strands or by comparing the consensus sequences of complementary strands. In some embodiments, the invention comprises after the sequencing step, a step of sequence read alignment and a step of generating a consensus sequence. In some embodiments, consensus is a simple majority consensus described in U.S. Patent 8535882. In other embodiments, consensus is determined by Partial Order Alignment (POA) method described in Lee et al. (2002) “Multiple sequence alignment using partial order graphs,” Bioinformatics, 18(3):452-464 and Parker and Lee (2003) “Pairwise partial order alignment as a supergraph problem—aligning alignments revealed,” J. Bioinformatics Computational Biol., 11:1-18. Based on the number of iterative reads used to determine a consensus sequence, the sequence may be largely free or substantially free of errors.

### EXAMPLES

### Example 1. Preparing Gapped-Circle Templates by PCR with “Handle” Primers

In this example, preparation of the gapped-circle templates commenced with amplification of the target nucleic acid with amplification primers comprising a 5′ “handle” or 5′ sequence including the nicking sites.

The initial PCR with target-specific primers included pUC19 plasmid, 5× reaction buffer, dNTPs, Forward primer, Reverse primer consisting of a target-specific sequence and a 5′-handle (Table 1, Nb.BsrDI recognition sequence highlighted), Q5 polymerase (New England BioLabs) and water. The PCR took place under the standard thermocycling profile and PCR products were purified with Ampure XP beads (Beckman Coulter) according to the manufacturer's recommendations.

TABLE 1
Primers and blocking oligonucleotides

| SEQ ID NO | Name | Sequence |
|---|---|---|
| SEQ ID NO: 1 | Forward | TGCCCATTGCGGAGGAGG |
| | primer | AGGACATTGC-target |
| | | sequence |
| SEQ ID NO: 2 | Reverse | AATGCAATGTGTGGTAGC |
| | primer | AATGGATCC-target |
| | | sequence |
| SEQ ID NO: 3 | Handle_F | /5-phos/TGCCCATTGC |
| | | GGAGGAGGAGGACATTGC |
| SEQ ID NO: 4 | Handle_R | /5-phos/AATGCAATGT |
| | | GTGGTAGCAATGGATCC |
| SEQ ID NO: 5 | Oligo_1 | TCCTCCTCCTCCGCAATG |
| SEQ ID NO: 6 | Oligo_2 | GGCAAATGCAATG |
| SEQ ID NO: 7 | Oligo_3 | TGTGGTAGCAATG |

The second “handle” PCR with 5′phosphate-modified handle-only primers included amplicon from pre-PCR, 5× reaction buffer, dNTPs, forward and reverse handle primers consisting of a handle sequence and a 5′phosphate (Table 1), Q5 polymerase and water. The PCR took place under the standard thermocycling profile and PCR products were purified with Ampure XP beads according to the manufacturer's recommendations.

For self-ligation, the amplicon from the second PCR step was diluted to 6 ng/μl and then mixed with 8× Volume ligation mix and distributed among eight 2-mL tubes, each containing 360 μL. The ligation mixture contained Blunt/TA ligase master mix (New England BioLabs) and was incubated at 20C for 60 minutes. Following the ligaton, the reactions were incubated with ExoIII (New England BioLabs) at 37 C for 60 minutes.

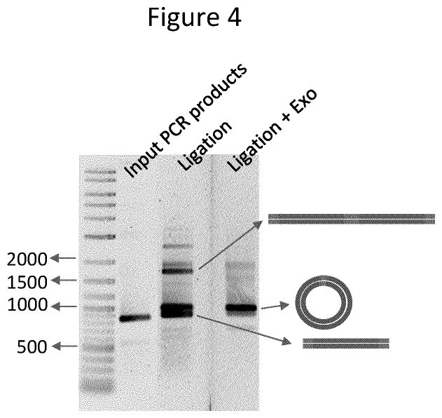

The circularized DNA was subjected to column purification using the QIAquick PCR purification kit according to manufacturer's recommendations and reconstituted in water. The efficiency of circularization and ExoIII treatment were assessed by gel electrophoresis (FIG. 4).

Digestions with the nicking enzyme Nb.BsrDI (New England BioLabs) was conducted in the CutSmart buffer according to the manufacturer's recommendations. The nicked dsDNA circles were purified using the QIAquick column.

The fragments resulting from nicking the DNA were removed by two rounds of heat denaturation in the presence of a 100-fold molar excess of competitor oligonucleotides (Table 1) in annealing buffer (100 mM TrisHCl, 500 mM NaCl, 10 mM EDTA). After each round of heat denaturation, the DNA was purified using the QIAquick column.

The efficiency of forming gapped circles was tested by digestion with a Type II restriction enzyme BsrDI (New England BioLabs). Digestion products were run on a 1% TAE agarose and run at 200 V for 20 mins. Results are shown on FIG. 4. As predicted, the dsDNA circle is cut by BsrDI while the gapped DNA is not cut by BsrDI.

A biotinylated threading blocker primer was ligated into the gap of the gapped circle using the ligase in a ligase buffer according to the manufacturer's protocol. The ligation products were purified with the QIAquick column and analyzed by BsrDI digestion and gel electrophoresis. As shown in FIG. 4, the gapped ds circle with the ligated oligo is partially digested by BsrDI.

The gapped circles with ligated threading blocker were sequenced on a biological nanopore instrument. The resulting accuracy was 84% and median processing length ranged between 5.6 kb and 6 kb.

### Example 2. Preparing Gapped-Circle Templates by Ligating Adaptors

In this example, the first step was ligation of adaptors comprising the Nb.BsrDI. The subsequent steps of forming the gapped ds circles including the steps of “handle PCR,” self-ligation, exonuclease treatment, Nb.BsrDI digestion, gap formation and ligation of the blocker oligonucleotide were performed as described in Example 1.

1. A method of forming a gapped circle nucleic acid template, wherein the method comprises the following steps:
(a) attaching an adaptor to at least one end of a double-stranded nucleic acid in a sample forming an adapted nucleic acid, wherein only one strand of the adaptor comprises a cleavage site;
(b) joining the ends of the adapted nucleic acid to form a circular adapted nucleic acid; and
(c) contacting the circular adapted nucleic acid with a cleaving agent recognizing the cleavage site to remove a portion of only one strand in the circular adapted nucleic acid, thereby forming a gapped circle nucleic acid template having a circular strand and a gapped strand.

2. The method of claim 1, wherein the adaptor is attached by extending a primer comprising a target-specific sequence and the adaptor sequence.

3. The method of claim 1, wherein the adaptor is attached by ligation.

4. The method of claim 1, wherein the adaptor comprises a nucleic acid barcode.

5. The method of claim 1, wherein the cleaving agent is a nicking endonuclease and the cleavage site is the nicking endonuclease recognition site.

6. The method of claim 1, further comprising a step of amplifying the adapted nucleic acid prior to forming the circular adapted nucleic acid.

7. The method of claim 1, wherein the cleaving agent is uracil-N-Glycosylase and the cleavage site is a uridine-containing nucleotide.

8. The method of claim 1, further comprising a step of contacting the sample with an exonuclease after the step of forming the circular adapted nucleic acid.

9. The method of claim 1, wherein in step (b), joining the ends of the adapted nucleic acid to form a circular adapted nucleic acid is by ligation.

10. The method of claim 1, wherein in step (c), removing the portion of only one strand in the circular adapted nucleic acid is by heat denaturation after cleavage with the cleaving agent.

11. The method of claim 1, wherein the circular strand comprises a primer-binding site in the gap portion of the gapped circle.

12. The method of claim 1, wherein the gapped strand of the gapped circle comprises an extendable 3′-end.

13. The method of any one of claims 1-12, further comprising sequencing the target nucleic acid by extending the extendable 3′-end to copy at least a portion of the circular strand.

14. A method of sequencing nucleic acids in a sample, wherein the method comprises the following steps:
(a) forming a library of gapped circle nucleic acid templates, wherein the method comprises the following steps:
(i) attaching an adaptor to at least one end of double stranded nucleic acids in a sample, thereby forming adapted nucleic acids, wherein only one strand of the adaptor comprises a cleavage site and the adaptor comprises a primer binding site;
(ii) joining the ends of each of the adapted nucleic acids to form circular adapted nucleic acids; and
(iii) contacting the circular adapted nucleic acids with a cleaving agent that recognizes the cleavage site to remove a portion of only one strand in each of the circular adapted nucleic acids, thereby forming a library of gapped circle nucleic acid templates having a gapped strand with an extendable 3′-end and a circular strand; and
(b) extending the extendable 3′-end to copy at least a portion of the circular strand, thereby sequencing the library of gapped circle nucleic acid templates by a sequencing-by-synthesis method.

15. A method of forming a library of gapped circle nucleic acid templates, wherein the method comprises the following steps:
(a) attaching an adaptor to at least one end of double-stranded nucleic acids in a sample, thereby forming adapted nucleic acids, wherein one strand of the adaptor comprises a cleavage site;
(b) joining the ends of each of the adapted nucleic acids to form circular adapted nucleic acids; and
(c) contacting the circular adapted nucleic acids with a cleaving agent that recognizes the cleavage site to remove a portion of only one strand in each of the circular adapted nucleic acids, thereby forming a library of gapped circle nucleic acid templates.

16. A method of forming an enriched library of gapped circle nucleic acid templates, wherein the method comprises the following steps:
(a) attaching an adaptor to at least one end of double stranded nucleic acids in a sample, thereby forming adapted nucleic acids,
(b) hybridizing to the adapted nucleic acids a first target-specific primer, wherein the first target-specific primer has a capture moiety;
(c) capturing the adapted nucleic acid hybridized to the first primer via the capture moiety, thereby enriching the target nucleic acids;
(d) hybridizing to the enriched adapted target nucleic acids a second primer, wherein the second primer comprises a sequence of one or more cleavage sites;
(e) extending the second primer to form a double-stranded adapted nucleic acid with one or more cleavage sites on only one strand;
(f) joining the ends of each of the double-stranded nucleic acids to form circular adapted nucleic acids; and
(g) contacting the circular adapted nucleic acids from step (f) with a cleaving agent that recognizes the cleavage site to remove a portion of only one strand in each of the circular adapted nucleic acids, thereby forming a library of enriched gapped circle nucleic acid templates.

17. A method of forming an enriched library of gapped circle nucleic acid templates, wherein the method comprises the following steps:
(a) attaching an adaptor to at least one end of double stranded nucleic acids in a sample, thereby forming adapted nucleic acids;
(b) hybridizing a first target-specific primer to the adapted nucleic acids, wherein the first target-specific primer comprises a capture moiety;
(c) capturing the adapted nucleic acid hybridized to the first primer via the capture moiety;
(d) hybridizing a second primer to the captured adapted nucleic acid, wherein the second primer hybridizes to the same strand as the first primer;
(e) extending the hybridized second primer, thereby producing a double-stranded adapted nucleic acid and displacing the first primer comprising the capture moiety;
(f) hybridizing a third primer to the adaptor within the adapted nucleic acids hybridized to the second primer, wherein the third primer comprising a sequence of one or more cleavage sites;
(g) extending the third primer, thereby forming a double-stranded adapted nucleic acid with one or more cleavage sites;
(h) joining the ends of each of the double-stranded adapted nucleic acid with one or more cleavage sites to form circular adapted nucleic acids; and
(i) contacting the circular adapted nucleic acids from step (h) with a cleaving agent that recognizes the cleavage site to remove a portion of one strand in each of the circular adapted nucleic acids, thereby forming a library of enriched gapped circle nucleic acid templates.
